(19) United States
(12) Patent Application Publication
(10) Pub. No.: US 2017/0189447 A1
(43) Pub. Date: Jul. 6, 2017

(54) FORMULATION FOR INCREASING ENERGY

(71) Applicant: NIS Clinical Research, Kaysville, UT (US)

(72) Inventor: Steven Ray Morris, Layton, UT (US)

(73) Assignee: NIS Clinical Research, Kaysville, UT (US)

(21) Appl. No.: 15/398,598

(22) Filed: Jan. 4, 2017

Related U.S. Application Data
(60) Provisional application No. 62/275,117, filed on Jan. 5, 2016.

(51) Int. Cl. A61K 33/34; A61K 31/122; A61K 31/465; A61K 31/525; A61K 31/205; A61K 33/26; A61K 33/32; A61K 33/24; A61K 33/30; A61K 31/475; A61K 33/06

(52) U.S. Cl. CPC A61K 33/34; A61K 31/475; A61K 31/122; A61K 31/465; A61K 31/525; A61K 33/06; A61K 33/26; A61K 33/32; A61K 33/24; A61K 33/30; A61K 31/205

(57) ABSTRACT

A composition is disclosed that includes formulation A, wherein formulation A comprises pyrroloquinoline quinone (PQQ), coenzyme Q (CoQ10), niacinamide and riboflavin in a combined amount sufficient to increase mitochondrial mass in a cell. In some embodiments, the composition also includes formulation B, wherein formulation B comprises a magnesium chelate, iron, a copper salt, a manganese salt, a molybdenum salt, a zinc salt, and organic selenium. The disclosed compositions are of use for increasing mitochondrial biogenesis in a cell of a subject, increasing the activity level or exercise capacity of a subject, and/or increasing alertness and/or cognition.

### PRIORITY CLAIM

This application claims the benefit of U.S. Provisional Application No. 62/275,117, filed Jan. 5, 2016, which is herein incorporated by reference in its entirety.

### FIELD

This relates to the field of nutraceuticals, specifically to compositions including pyrroloquinoline quinone (PQQ), coenzyme Q (CoQ10), niacinamide and riboflavin in a combined amount sufficient to increase mitochondrial mass in a cell.

### BACKGROUND

The mitochondria, found in cells of mammals, is the main organelle involved in energy production. Mitochondria provide direct and indirect biochemical regulation of a wide array of cellular respiratory, oxidative and metabolic processes. These processes include electron transport chain (ETC) activity, which drives oxidative phosphorylation to produce metabolic energy in the form of adenosine triphosphate (ATP).

In addition to their role in energy production in growing cells, mitochondria participate in apoptosis (Newmeyer et al., Cell 79:353-364, 1994; Liu et al., Cell 86:147-157, 1996). Altered or defective mitochondrial activity, including but not limited to failure at any step of the ETC, may result in the generation of highly reactive free radicals that have the potential of damaging cells and tissues. Mitochondrial defects, which include defects related to the discrete mitochondrial genome that resides in mitochondrial DNA and/or to the extramitochondrial genome, contribute significantly to the pathogenesis of diseases associated with altered mitochondrial function. A number of degenerative, hyperproliferative and other types of diseases are thought to be caused by, or to be associated with, alterations in mitochondrial function. These include, but are not limited to, Alzheimer's disease, Parkinson's disease, Huntington's disease, diabetes mellitus, and hyperproliferative disorders, such as cancer, tumors and psoriasis, amyotrophic lateral sclerosis (ALS), Friedreich's ataxia, colon cancer, and exercise intolerance. Increased mitochondrial biogenesis and increased anti-oxidant capacity also plays a role in exercise capacity. Thus, there is a need for methods to increase anti-oxidant capacity and exercise capacity in subjects.

### SUMMARY

A composition is disclosed that includes formulation A, wherein formulation A comprises pyrroloquinoline quinone (PQQ), coenzyme Q (CoQ10), niacinamide and riboflavin in a combined amount sufficient to increase mitochondrial mass in a cell. In some embodiments, the composition also includes formulation B, wherein formulation B comprises a magnesium chelate, iron, a copper salt, a manganese salt, a molybdenum salt, a zinc salt, and organic selenium.

In additional embodiments, methods are also disclosed for increasing mitochondrial biogenesis in a cell of a subject using the disclosed compositions.

In some embodiments, methods are disclosed for increasing the activity level or exercise capacity of a subject. In other embodiments, methods are disclosed for increasing alertness and/or cognition using the disclosed compositions.

The foregoing and other objects, features, and advantages of the invention will become more apparent from the following detailed description, which proceeds with reference to the accompanying figures.

### BRIEF DESCRIPTION OF THE DRAWINGS

FIG. 1. Results of the Folin-Ciocalteu assay. A serial dilution series (2-fold) of the product was tested and results are presented in gallic acid equivalents (GAE) per gram.

FIG. 2. Relative mitochondrial mass per cell in 2-hour cultures, lymphocyte subset. The bar graph plots MITOTRACKER® Green mean fluorescence intensity. Statistical significance is indicated as ** p<0.01.

FIG. 3. Relative mitochondrial mass per cell in 2-hour cultures, monocyte subset. The bar graph plots MITOTRACKER® Green mean fluorescence intensity. Statistical significance is indicated as ** p<0.01.

FIG. 4. Relative mitochondrial mass per cell in 2-hour cultures, PMN cell subset. The bar graph plots MITOTRACKER® Green mean fluorescence intensity. Statistical significance is indicated as ** p<0.01.

FIG. 5. Study protocol. Volunteers are tested on separate clinic days with a 7-day wash-out period between the two initial test days, and again 4 weeks after the second test day. The sequence for testing of acute effects of active product and placebo, as presented, is an example, since the order in which products are fed to study participants for acute effects will be randomized.

### DETAILED DESCRIPTION

Disclosed are compositions that increase mitochondrial mass and/or mitochondrial biogenesis in a cell. These compositions include or consist of formulation A, which includes or consists of pyrroloquinoline quinone (PQQ), coenzyme Q (CoQ10), niacinamide and riboflavin in a combined amount sufficient to increase mitochondrial mass in a cell. Formulation A provides an unexpected increase in mitochondrial mass and/or antioxidant activity. In some embodiments, formulation A is combined with Formulation B, which includes or consists of a magnesium chelate, iron, a copper salt, a manganese salt, a molybdenum salt, a zinc salt, and organic selenium.

The disclosed compositions are of use for increasing the activity level, exercise capacity or stamina of a subject. They are also of use for increasing alertness and cognition. In some embodiments, the composition activates the Nuclear respiratory factor (Nrf)-1 and Nrf-2 pathways, activates peroxisome proliferator-activated receptor γ coactivator 1 (PGC1) pathway, and/or increases phospholipase, cyclooxygenase and/or lipooxygenase pathways in the cell. In other embodiments, the composition inhibits the release or production of an interleukin, leukotriene, tumor necrosis factor, C-reactive protein, and/or an inflammatory cytokine in a subject.

### Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. As used herein, “comprises” means “includes.” Thus, “comprising A or B,” means “including A, B, or A and B,” without excluding additional elements.

Administration: To provide or give a subject an agent, such as the disclosed compositions, by any effective route. Exemplary routes of administration include, but are not limited to, oral, injection (such as subcutaneous, intramuscular, intradermal, intraperitoneal, intravenous, and intraperitoneal), sublingual, transdermal, intranasal, topical and inhalation routes.

Biological sample: A sample of biological material obtained from a subject. Biological samples include all clinical samples useful for detection of disease (such as Parkinson's disease) in subjects. Appropriate samples include any conventional biological samples, including clinical samples obtained from a human or veterinary subject. Exemplary samples include, without limitation, cells, cell lysates, blood samples, white blood cell samples, plasma samples, leukocytes and platelets. In a particular example, a biological sample is obtained from a subject having, suspected of having or at risk of having, a disease.

Cytokines: Small proteins that are important in cell signaling. Cytokines are produced by a broad range of cells, including immune cells such as macrophages, lymphocytes and mast cells. Cytokines include chemokines, interferons, interleukins, lymphokines and tumor necrosis factor alpha Inflammatory cytokines are a type of cytokine that promotes systemic inflammation Inflammatory cytokines (often referred to as “proinflammatory cytokines”) include, but are not limited to, IL-1β, IFN-γ, IL-6, IL-8, and TNF-α.

Detecting: To identify the existence, presence, or fact of something. General methods of detecting are known to the skilled artisan and may be supplemented with the protocols and reagents disclosed herein. For example, included herein are methods of detecting mitochondrial mass. Detection can include a physical readout, such as fluorescence or a reaction output.

Effective Amount: An amount of a composition that alone, or together with an additional agent(s) (for example additional antioxidants), induces the desired response. The preparations disclosed herein can be administered in therapeutically (for example nutraceutically) effective amounts. The effective amount can be administered in a single dose, or in several doses, for example daily. However, the effective amount can depend on the subject being treated, the severity and type of the condition being treated, and the manner of administration.

Isolated: An “isolated” biological component (such as a nucleic acid molecule, protein, or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Mitochondrion: A double membrane-bound organelle found in most eukaryotic cells that ranges in size from 0.5 to 1.0 μm in diameter. Mitochondria generate most of the cell's supply of adenosine triphosphate (ATP. In addition to supplying cellular energy, mitochondria are involved in other tasks, such as signaling, cellular differentiation, and cell death, as well as maintaining control of the cell cycle and cell growth. A mitochondrion has its own genome, independent from the cellular genome in the nucleus.

Mitochondrial biogenesis: A processes of growth, amplification and healthy maintenance of the mitochondria. Mitochondria produce ATP in a cell. Mitochondrial biogenesis is a complex process involving both nuclear and mitochondrial players. Mitochondrial DNA encodes a small number of proteins, which are translated on mitochondrial ribosomes. Most of these proteins are highly hydrophobic subunits of the respiratory chain, which is localized in the inner mitochondrial membrane. Nuclear-encoded proteins are translated on cytosolic ribosomes and imported into mitochondria. These proteins include structural proteins, enzymes or enzyme subunits, components of the import-, replication-, transcription- and translation-machinery and chaperones. Niacinamide: An amide of nicontinic acid, also known as nicotinamide.

Niacinamide is a water-soluble vitamin and is part of the vitamin B group. Nicotinic acid, also known as niacin, is converted to nicotinamide in vivo, and, though the two are identical in their vitamin functions, nicotinamide does not have the same pharmacological and toxic effects of niacin, which occur incidental to niacin's conversion.

Nutrients: Nutrients are nutritious components in foods that an organism uses to survive and/or thrive. Although nutrients are present in food, a nutrient as used herein can be either naturally occurring or synthetically manufactured. A “whole food nutrient” refers to a nutrient that is found in whole food and not synthetically made. Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

Pharmaceutically Acceptable Carrier: An inactive substance used as a carrier for the active ingredients of a composition. Excipients can include substances that are used to bulk up formulations with very potent active ingredients, allow for convenient and accurate dosage, stabilize the active ingredients, and make the delivery system optically and/or organoleptically acceptable. Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like.

The pharmaceutically acceptable vehicles (carriers) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more compositions. The use of pharmaceutically acceptable carriers does not imply that that product so made is useful only for pharmaceutical purposes. Rather it implies that the product is suitable for administration to or consumption by a subject, for example as a pharmaceutical or nutraceutical that is suitable for oral ingestion by a subject.

In general, the nature of the vehicle will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid vehicles can include, for example, pharmaceutical grades of mannitol, lactose, starch, or magnesium stearate. In addition to biologically-neutral vehicles, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically acceptable salt: A biologically compatible salt of a compound that can be used as a drug, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.)

Purified: The term purified does not require absolute purity; rather, it is intended as a relative term. Thus, for example, a purified substance is one in which the substance is more enriched than the substance in its natural environment. In one embodiment, a preparation is purified such that the substance represents at least about 5% (such as, but not limited to, at least 10%, 20%, 30%, 40%, 50%, 70%, 80%, 90%, 95%, 98% or 99%) of the total content of the preparation, such as by weight.

Range: With respect to ranges, the term “in the range of x to y” or “from x to y” includes any value between x and y, as well as the endpoints x and y.

Riboflavin (vitamin B2): A small molecule, also known as vitamin B2 or 7,8-Dimethyl-10-[(2S,3S,4R)-2,3,4,5-tetrahydroxypentyl]benzo[g]pteridine-2,4-dione, that is essential to human nutrition.

Subject: Living multi-cellular vertebrate organisms, a category that includes both human and veterinary subjects, such as a companion animal, including a cat, dog or horse. A “subject in need of an increase in mitochondrial biogenesis and/or antioxidant activity” is a subject who may benefit from such an increase, such as a subject who desires to decrease signs of age, offset tissue damage caused by oxidation, and/or improve cardiovascular, neurological, tumor-related, skin-appearance or other conditions that are associated with oxidative stress.

Therapeutically effective dose: An amount sufficient to provide a beneficial, or therapeutic, effect to a subject or a given percentage of subjects.

Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

Unit dose: A physically discrete unit containing a predetermined quantity of an active material calculated to individually or collectively produce a desired effect, such as a therapeutic effect. A single unit dose or a plurality of unit doses can be used to provide the desired effect or activity, such as antioxidant activity. In one example, a unit dose includes a desired amount of an agent that promotes cardiovascular or cognitive health. In another example, the unit dosage form contains multiple predetermined dosages of the active material.

It is to be understood that all base sizes or amino acid sizes, and all molecular weight or molecular mass values, given for components are approximate, and are provided for description. All percentages and ratios are calculated by weight unless otherwise indicated. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Compositions: Formulation A

Disclosed herein is a composition comprising formulation A, wherein formulation A comprises pyrroloquinoline quinone (PQQ), coenzyme Q (CoQ10), niacinamide and riboflavin in a combined amount sufficient to increase mitochondrial mass in a cell.

In some embodiments, the composition includes about 5% to about 8% PQQ by weight percent of Formulation A. In further embodiments, the composition includes about 41% to about 43% CoQ10 by weight of Formulation A. In more embodiments, the composition includes about 41% to about 43% of niacinamide by weight of Formulation A. In yet other embodiments, the composition includes about 10% riboflavin by weight of Formulation A. Thus, in a specific non-limiting example, the composition includes about 5% to about 8% PQQ by weight percent of Formulation A, about 41% to about 43% CoQ10 by weight of Formulation A, about 41% to about 43% of niacinamide by weight of Formulation A, and about 9 to 12% riboflavin by weight of Formulation A, such as 10% riboflavin. In this context (percentage), “about” means within 0.5%.

The milligram amounts of pyrroloquinoline quinone (PQQ), coenzyme Q (CoQ10), niacinamide and riboflavin listed in the below paragraphs can be included in Formulation A in any combination. In non-limiting examples, formulation A can include:

| | |
|---|---|
| Ingredient | Range |

| | | |
|---|---|---|
| 1. | PQQ | 5-20 mg |
| 2. | CoQ10 | 45-100 mg |
| 3. | Niacinamide | 45-100 mg |
| 4. | Riboflavin | 10-25 mg |

In some embodiments, PQQ, CoQ10, niacinamide, and riboflavin are included in Formulation A at a ratio of about 1:5:5:1.2, respectively.

In one specific non-limiting example, Formulation A includes about 10 mg of PQQ, about 50 mg of CoQ10, about 50 mg of niacinamide, and about 12.5 mg of riboflavin. However, as discussed below, these amounts can be varied, provided the composition increases mitochondrial mass.

These components are discussed in more detail below. Without being bound by theory, formulation A can activate the PGC-1 pathway, activate the Nrf-1 and Nrf-2 pathways, up-regulate mitochondrial gene expression and protein synthesis, and/or increase mitochondrial biogenesis.

### Pyrroloquinoline Quinone

In some embodiments, formulation A includes about 5 to about 20 milligrams (mg) of PQQ, such as about 8 to about 15 mg of PQQ, or about 10 to about 12 mg of PQQ. Formulation A can include about 5, 6, 7, 8, 9, 10, 11, 12, 13, 14, 15, 16, 17, 18, 19 or 20 mg of PQQ. In this context (milligram amounts), throughout the specification “about” means within 1 mg.

In additional embodiments, the composition includes about 5% to about 8% PQQ by weight percent of formulation A. However, the composition can include, for example, about 3% to about 10% of PQQ by weight of formulation A. In specific non-limiting examples, the composition includes about 4% to 9% of PQQ by weight for formulation A. The composition can include for example, about 5%, about 6%, about 7% or about 8% of PQQ by weight of formulation A.

Pyrroloquinoline quinone (PQQ) is a water soluble anionic quinone that can transfer electrons catalytically between a variety of reductants and oxidants, and may be part of a soluble electron transport system in eukaryotic cells. PQQ proper is of the general structure

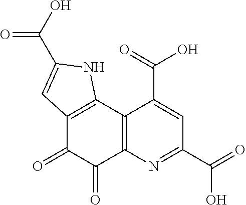 [SMILES: O=C(O)c1cc(C(=O)O)c2c(n1)C(=O)C(=O)c1cc(C(=O)O)[nH]c1-2]

“Pyrroloquinoline quinone” includes any member of the pyrroloquinoline quinone family having chemical similarity, including closely related isomeric and stereoisomeric analogs of PQQ (See e.g., Zhang et al., 1995, Biochem. Biophys. Res. Commun. 212: 41-47, 1995). PQQ is also known as methoxatin. PQQ is found in animal tissues and fluids. Without wishing to be bound by theory, PQQ may act in part as a free-radical scavenger, particularly of reactive nitrogen species (RNS) and reactive oxygen species (ROS). PQQ can be included as a pharmaceutically acceptable salt, such as a calcium salt, see for example, U.S. Pat. No. 8,969,563, which is incorporated by reference herein. The PQQ can be included as a disodium salt, see for example, Published U.S. Patent Application No. 2014/0128609, which is incorporated herein by reference. In one non-limiting example, the PQQ is a disodium salt.

### Coenzyme Q (CoQ)

In additional embodiments, formulation A includes about 45 to about 100 mg of CoQ10, such as about 47 to about 75 mg of CoQ, about 50 to about 70 mg of CoQ, or about 50 to about 60 mg of CoQ. Formulation A can include, for example, about 45, about 50, about 55, about 60, about 65, about 70, about 75, about 80, about 85, about 90, about 95 or about 100 mg of CoQ.

In some embodiments, the composition includes about 40% to about 45% of CoQ by weigh of Formulation A, such as about 41% to about 43% CoQ10 by weight of formulation A. Thus, the composition can include, for example, about 41%, about 42% or about 43% of CoQ10 by weight of formulation A.

Coenzyme Q10 in its various forms is also called CoQ10, ubiquinone, ubidecarenone, ubiquinol and vitamin Q10. Coenzyme Q compounds are benzoquinone compounds containing isoprenyl units. The number of isoprenyl units in each of the different CoQ species is indicated with a number following CoQ. Coenzyme Q can exist in two different forms: an oxidized form and a reduced form. When the oxidized form of a Coenzyme Q species is reduced by one equivalent, it becomes a ubisemiquinone, denoted QH, which contains a free radical on one of the oxygens in the benzene ring of the benzoquinone. Oxidized and reduced coenzyme Q containing compounds can be used as active ingredients in the provided compositions.

Coenzyme is shown below.

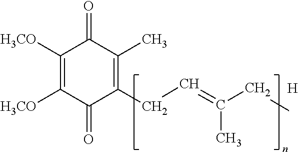

The “Q” in the name refers to Quinone and the 10 refers to the number of isoprenoid units. Thus, in the structure above for Coenzyme Q, n=10.

Coenzyme Q10 (CoQ10) is a benzoquinone compound that contains 10 isoprenoid units. CoQ10 known as 2-[(2E,6E,10E,14E,18E,22E,26E,30E,34E)-3,7,11,15,19,23,27,31,35,39-Decamethyltetraconta-2,6,10,14,18,22,26,30,34,38-decaenyl]-5,6-dimethoxy-3-methylcyclohexa-2,5-diene-1,4-dione. The structure of

In one embodiment, CoQ10 refers to the oxidized form of CoQ10, which also is referred to as ubidecarenone, as opposed to the reduced form of CoQ10. As noted above, both the reduced and/or oxidized CoQ10 are exemplary of the coenzyme Q species that can be used as active ingredients in the provided compositions.

CoQ10 has electron-transfer ability and is present in cellular membranes, such as those of the endoplasmic reticulum, peroxisomes, lysosomes, vesicles and the mitochondria. A decrease in natural CoQ10 synthesis has been observed in sick and elderly people.

CoQ10 containing compounds are available commercially. Any CoQ10 compound or reduced CoQ10 compound can be used in formulation A. Exemplary of the CoQ10 compounds that can be used as active ingredients are coenzyme Q10 compounds containing greater than 98% or greater than about 98% ubidecarenone, for example, the compound sold under the name KANEKA Q10™ (USP Ubidecarenone) by Kaneka Nutrients, L.P., Pasadena, Tex. The compound sold under the name KANEKA Q10™ is fermented entirely from yeast and is identical to the body's own CoQ10 and free from the cis isomer found in some synthetically produced CoQ10 compounds. In one example, a fermented non-CIS-isomer of CoQ10 is utilized.

### Niacinamide

In further embodiments Formulation A includes about 45 to about 100 mg of niacinamide. Formulation A can include about 47 to about 75 mg of niacinamide, about 50 to about 70 mg of niacinamide, or about 50 to about 60 mg of niacinamide. Formulation A can include, for example, about 45, about 50, about 55, about 60, about 65, about 70, about 75, about 80, about 85, about 90, about 95 or about 100 mg of niacinamide.

In some embodiments, the composition includes about 35% to about 50%, such as about 40% to about 45% of niacinamide by weigh of Formulation A, for example about 41% to about 43% niacinamide by weight of formulation A. In some embodiments, the composition can include, for example, about 40%, about 41%, about 42%, about 43%, about 44%, or about 45% of niacinamide by weight of formulation A.

Niacinamide is also known as as nicotinamide and nicotinic amide. The structure of naicinamide is shown below:

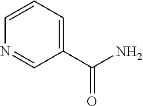

### Riboflavin

In some embodiments, Formulation A includes about 10 to about 25 mg of riboflavin, such as about 11 to about 20 mg of riboflavin, or about 12.5 to about 15 mg of riboflavin. Formulation A can include, for example, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 mg of riboflavin.

In some embodiments, Formulation A includes about 9% to about 12% riboflavin by weight of formulation A, such as about 10 to 12% riboflavin by weight of Formulation A, for example about 10% riboflavin by weight of Formulation A.

Riboflavin has the formula C₁₇H₂₀N₄O₆, and it's IUPAC ID is 7,8-Dimethyl-10-[(2 S,3S,4R)-2,3,4,5-tetrahydroxypentyl]benzo[g]pteridine-2,4-dione. Riboflavin can be produced commercially by several processes, see U.S. Pat. No. 7,670,800 and U.S. Pat. No. 5,095,115, and U.S. Pat. No. 5,103,005, which are incorporated herein by reference.

Riboflavin derivatives, which are also of use in Formulation A, include flavin mononucleotide, flavin adenine dinucleotide, riboflavin tetraacetate, riboflavin tetrabutyrate, and derivatives in which various hydroxyl groups of the ribitol have been derivatized, and other isoalloxazine derivatives (including proflavin, lumiflavin, lumiflavin-3-acetate, lumi-chrome, and derivatives in which chemical groups are substituted on the various carbon and nitrogen atoms of the isoalloxazine ring. Riboflavin can be obtained commercially from Sigma Chemical Co., St. Louis, Mo.

### Compositions: Additional Ingredients and Formulation B

The composition can include additional active agents. In some embodiments, the composition includes one or more of a magnesium chelate, iron, a copper salt, a manganese salt, a molybdenum salt, a zinc salt, and organic selenium, in an amount sufficient to increase mitochondrial function. In a specific non-limiting example, the composition can include one or more of about 20 to about 50 mg of the magnesium chelate, about 10 to about 30 mg of iron, about 1 mg to about 4 mg of copper salt, about 1 mg to about 4 mg of manganese salt, about 0.05 to about 0.2 mg of the molybdenum salt, about 10 mg to about 30 mg of a zinc salt; and about 0.1 mg to about 0.4 mg of organic selenium. In non-limiting examples, the composition can include about 25 mg of the magnesium chelate, about 15 mg of iron, about 2 mg of copper salt, about 2 mg of manganese salt, about 15 mg of a zinc salt, about 0.1 mg of the molybdenum salt; and/or about 0.2 mg of organic selenium. The selenium can be selenomethionine or selenocysteine.

In some embodiments, the composition includes Formulation B, wherein Formulation B comprises the magnesium chelate, the iron, the copper salt, the manganese salt, the molybdenum salt, the zinc salt, and the organic selenium. In further embodiments, Formulation B includes: about 42% to about 47% of the magnesium chelate by weight of Formulation B, about 23% to about 25% of the iron by weight of Formulation B, about 2% to about 3% of the copper salt by weight of Formulation B, about 2% to about 3% of the manganese salt by weight of Formulation B, about 0.1% to about 0.2% of the molybdenum salt by weight of Formulation B, about 23% to about 25% of the zinc salt by weight of Formulation B, and about 0.2% to about 0.3% of the organic selenium by weight of Formulation B. Each of these agents is discussed in more detail below, under specific headings.

In a specific non-limiting example, Formulation B can include about 20 to about 50 mg of the magnesium chelate, about 10 to about 30 mg of iron, about 1 mg to about 4 mg of copper salt, about 1 mg to about 4 mg of manganese salt, about 0.05 to about 2 mg of the molybdenum salt, about 10 mg to about 30 mg of a zinc salt; and about 0.1 mg to about 0.4 mg of organic selenium. In one embodiment, Formulation B includes all of:

| | |
|---|---|
| Ingredient | Range |

| | | |
|---|---|---|
| 1. | Magnesium Chelate (e.g., | 20-50 mg |
| | Krebs) | |
| 2. | Iron | 10-30 mg |
| 3. | Copper Citrate | 1-4 mg |
| 4. | Manganese Citrate | 1-4 mg |
| 5. | Molybdenum Citrate | 0.05-0.2 mg |
| 6. | Zinc Citrate | 10-30 mg |
| 7. | Organic methionine (e.g., | 0.1-0.4 mg |
| | selenomethionine) | |

In one specific non-limiting example, Formulation B comprises about 25 mg of the magnesium chelate, about 15 mg of iron, about 2 mg of copper salt, about 2 mg of manganese salt, about 15 mg of a zinc salt, about 0.1 mg of the molybdenum salt, and about 0.2 mg of organic selenium. In another non-limiting example, Formulation B includes about 25 mg of the magnesium chelate, about 15 mg of iron, about 2 mg of copper citrate, about 2 mg of manganese citrate, about 15 mg of a zinc citrate, about 0.1 mg of the molybdenum citrate, and about 0.2 mg of selenomethionine.

As discussed below, these amounts of these agents can be varied in the composition and in Formulation B, provided the composition increases mitochondrial mass. Without being bound by theory, Formulation B provides substrates and co-factors for electron transport complexes and the citric acid cycle. In addition, Formulation B can optimize carbohydrate catalysis, ATP production, and energy output.

Magnesium Chelate

The composition can include about 20 to about 50 mg of the magnesium chelate, such as in Formulation B. Thus, the composition can include about 22 mg to about 40 mg of the magnesium chelate, such as about 25 to about 35 mg of the magnesium chelate, such as about 25 to about 30 mg of the magnesium chelate, such as in Formulation B. In some embodiments, the composition includes about 20, about 21, about 22, about 23, about 24, about 25, about 26, about 27, about 28, about 29, about 30, about 31, about 32, about 33, about 34, or about 35 mg of the magnesium chelate, such as in Formulation B. In one specific non limiting example, the composition includes about 25 mg of the magnesium chelate, such as in Formulation B.

In some embodiments, the composition includes Formulation B, and Formulation B includes about 40% to about 48% of magnesium chelate, such as about 42% to about 47% of the magnesium chelate by weight of Formulation B. Thus, Formulation B can include be about 42%, about 43%, about 44%, about 45%, about 46%, or about 47% by weight of Formulation B.

A magnesium chelate is where the mineral salt is covalently bound with a protein amino acid. These typically result in magnesium complexes of aspartate, citrate, fumarate, gluconate, ketoglutarate, succinate, taurinate. Magnesium utilized in the disclosed compositions can be in a variety of forms and with various counter ions, including among others magnesium citrate, magnesium fumarate, magnesium gluconate, magnesium alpha-ketoglutarate, magnesium lactate, magnesium malate, magnesium succinate, magnesium picolinate, magnesium sulphate or mixtures thereof. In one embodiment, the magnesium is magnesium citrate, magnesium malate, magnesium malate-citrate, and magnesium (Krebs) in which the counter ions are a mixture of the anions of the five primary organic acids of the tricarboxylic acid cycle (Krebs Cycle) i.e., a mixture of the magnesium salts of citric, fumaric, malic, alpha-ketoglutaric and succinic acids, see PCT Publication No. 2000007607, which is incorporated herein by reference. Examples of U.S. Patents discussing the role of Magnesium and Magnesium Chelates of various types are seen in U.S. Pat. Nos. 5,270,297 and 5,292,538 and Published U.S. Patent Application No. 2004/0204382, incorporated herein by reference.

### Zinc Salts

The composition can include about 10 mg to about 30 mg of a zinc salt, such as in Formulation B. In some embodiments, the composition includes about 12 to about 25 mg of a zinc salt, for example about 15 to about 20 mg of the zinc salt, such as in Formulation B. The composition can include about 10, about 11, about 12, about 13, about 14, about 15, about 16, about 17, about 18, about 19 or about 20 mg of the zinc salt, such as in Formulation B. In a specific non-limiting example, the composition includes about 15 mg of the zinc salt.

In some embodiments, the composition includes Formulation B, and Formulation B includes about 20% to about 26% of the zinc salt by weight of Formulation B, such as 23% to about 25% of the zinc salt by weight of formulation B, such as about 23%, about 24%, about 25% of the zinc salt by weight of formulation B.

Zinc can be provided in a variety of forms and with various counter ions, including among others zinc citrate, zinc fumarate, zinc gluconate, zinc alpha-ketoglutarate, zinc lactate, zinc malate, zinc succinate, zinc picolinate or mixtures thereof. In one embodiment, the zinc salt, is zinc (Krebs) in which the counter ions are a mixture of the anions of the five primary organic acids of the tricarboxylic acid cycle (Krebs Cycle). Thus, the zinc salt can be a mixture of the zinc salts of citric, fumaric, malic, alpha-ketoglutaric and succinic acids.

### Iron

The composition can include about 10 mg to about 30 mg of iron, such as in Formulation B. In some embodiments, the composition includes about 12 to about 25 mg of iron, for example about 15 to about 20 mg of iron, such as in Formulation B. The composition can include about 10, about 11, about 12, about 13, about 14, about 15, about 16, about 17, about 18, about 19 or about 20 mg of iron, such as in Formulation B. In a specific non-limiting example, the composition includes about 15 mg of iron.

In some embodiments, the composition includes Formulation B, and Formulation B includes about 20% to about 26% of iron by weight of Formulation B, such as about 23% to about 25% of iron by weight of formulation B, such as about 23%, about 24%, about 25% of the iron by weight of formulation B.

Iron in a variety of forms can be used, see U.S. Pat. No. 8,802,114, which is incorporated herein by reference. A variety of iron compounds have been administered, including ferric and ferrous forms of elemental iron as salts, complexes, hydrates, chelates, and bound to polymer.

In some embodiments, the iron compound is an iron salt (e.g., a ferrous salt or a ferric salt), an iron chelate, an iron complex, or polymer bound iron. In certain embodiments, the iron compound is a ferric compound, as it may produce less side effects than ferrous compounds.

Iron salts include, but are not limited to, ferrous sulfate, ferrous gluconate, ferrous fumarate, ferric hypophosphite, ferric albuminate, ferric chloride, ferric citrate, ferric oxide saccharate, ferric ammonium citrate, ferrous chloride, ferrous iodide, ferrous lactate, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferrous ethylenediammonium sulfate tetrahydrate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, ferric acetate, ferric fluoride, ferric phosphate, ferric pyrophosphate, ferrous pyrophosphate, ferrous carbonate saccharate, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, ferric sodium pyrophosphate, ferrous carbonate, ferric hydroxide, ferrous oxide, ferric oxyhydroxide, ferrous oxalate, and/or combinations thereof.

Iron chelates and complexes include, but are not limited to, ferric pyrophosphate, soluble ferric pyrophosphate, iron polysaccharide, iron bis glycinate, iron proteinate, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-maltodextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N′-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, and/or combinations thereof.

### Copper Salt

The composition can include about 1 to about 4 mg of a copper salt, such as in Formulation B. In some embodiments, the composition can include about 2 mg to about 4 mg of a copper salt, for example about 2 mg to about 3 mg of a copper salt, such as in Formulation B. The composition can include about 1, about 1.5, about 2, about 2.5, about 3, about 3.5 or about 4 mg of a copper salt, such as in Formulation B. In a specific, non-limiting example, the composition includes 2 mg of a copper salt. The copper salt can be copper citrate.

In some embodiments, the composition includes Formulation B. The composition can include about 1% to about 4% of the copper salt by weight of Formulation B, such as about 2% to about 3% of the copper salt by weight of Formulation B, for example about 2%, about 2.25%, about 2.5%, about 2.75%, or about 3% of copper salt by weight of Formulation B. In one specific non-limiting example, the Formulation B includes 3% copper salt by weight for Formulation B.

The copper also can be any copper salt of interest, such as copper carbonate, copper citrate, copper gluconate, or copper sulfate. In a specific, non-limiting example, the copper is copper citrate. The copper can also be cupric oxide, copper sulfate, or a copper amino acid chelate. Suitable amino acid chelates include copper-lysine, copper-glycine or copper-methionine complex.

### Manganese Salt

The composition can include about 1 to about 4 mg of a manganese salt, such as in Formulation B. In some embodiments, the composition can include about 2 mg to about 4 mg of a manganese salt, for example about 2 mg to about 3 mg of a manganese salt, such as in Formulation B. The composition can include about 1, about 1.5, about 2, about 2.5, about 3, about 3.5 or about 4 mg of a manganese salt, such as in Formulation B. In a specific, non-limiting example, the composition includes 2 mg of a manganese salt. The manganese salt can be manganese citrate.

In some embodiment, the composition includes Formulation B. The composition can include about 1% to about 4% of the manganese salt by weight of Formulation B, such as bout 2% to about 3% of the manganese salt by weight of Formulation B, for example about 2%, about 2.25%, about 2.5%, about 2.75%, or about 3% of manganese salt by weight of Formulation B. In one specific non-limiting example, the Formulation B includes 3% manganese salt by weight for Formulation B.

The manganese salt can be any manganese salt of interest, including manganese chloride, manganese citrate, manganese gluconate, manganese glycerophosphate, manganese hypophosphite, Manganese sulfate, and manganese ascorbate. Manganous oxide can also be utilized.

### Molybdenum Salt

The composition can include about 0.05 to about 0.2 mg of the molybdenum salt, such as about 0.05 to about 0.15 mg of the molybdenum salt, for example, about 0.075 to about 0.1 mg of the molybdenum salt, for example about 0.1 mg of the molybdenum salt, such as in Formulation B. The composition can include 0.05, 0.075, 0.1, 0.15 or 0.2 mg of the molybdenum salt, such as in Formulation B. In a specific non-limiting example, the composition can include about 0.1 mg of molybdenum salt, such as molybdenum citrate.

In some embodiment, the composition includes Formulation B. The composition can include about 0.1% to about 0.3% of the molybdenum salt by weight of Formulation B, such as about 0.1% to about 0.25% of the molybdenum salt by weight of Formulation B, for example about 0.15% to about 0.2% of the molybdenum salt by weight of formulation B, such as about 0.2% by weight of Formulation B.

The molybdenum salt can be molybdenum citrate, or a molybdenum acid salts as ammonium molybdate, ammonium molybdate tetrahydrate, potassium molybdate, sodium molybdate, sodium molybdate di-hydrate and mixtures thereof.

### Organic Selenium

The composition can include about 0.1 mg to about 0.4 mg of organic selenium, such as in Formulation B. In some embodiments, the composition can include about 0.2 mg to about 0.44 mg of a organic selenium, for example about 2 mg to about 3 mg of a organic selenium, such as in Formulation B. The composition can include about 1, about 1.5, about 2, about 2.5, about 3, about 3.5 or about 4 mg of organic selenium, such as in Formulation B. In a specific, non-limiting example, the composition includes 2 mg of organic selenium. The organic selenium can be selenomethione or selenocysteine.

In some embodiment, the composition includes Formulation B. The composition can include about 0.2% to about 0.4% of organic selenium, such as 0.2% to about 0.23% of the organic selenium by weight of Formulation B, for example about 0.2%, about 0.225%, about 0.25%, about 0.275%, or about 0.3% of organic selenium by weight of Formulation B. In one specific non-limiting example, the Formulation B includes 0.3% organic selenium by weight.

Any organic selenium can be utilized in the present compositions. Exemplary organic selenium compounds include selenium compounds of cysteine and methionine, as well as an organic selenium compound such as RSeH, RSeR, RSeR′, RSeSeR and RSeSeR′, wherein R and R′ are the same or different and each is an aliphatic residue containing at least one reactive group selected from the group consisting of aldehyde, amino, alcoholic, carboxylic, phosphate, sulfate, halogen or phenolic reactive groups and combinations thereof, see U.S. Published Patent Application No. 2009/0191283.

Selenium-containing compounds can be provided in a variety of forms. Selenium also can be present in elemental form or as inorganic selenium. It is also noted that selenium occurs in different valence forms. For example, selenium compounds occur in which the selenium has a +4 valance or a +6 valence, as the selenite and selenate ions, respectively. Among the inorganic selenite and selenate forms are water soluble alkali metal salts thereof, and particularly, the sodium and potassium salts, sodium and potassium selenite and selenate. In specific non-limiting examples, sodium selenite and/or potassium selenite can be included in the composition, such as in Formulation B.

### Compositions: Formulations and Carriers

Generally, the methods disclosed use compositions that are formulated for pharmaceutical use. Pharmaceutically acceptable carriers include, but are not limited to, physiological saline, Ringer's, phosphate solution or buffer, buffered saline, and other carriers known in the art.

The route of administration may be one or more of buccal (via membranes of the mouth), oral, or enteral (via the gastrointestinal tract). The pharmaceutically acceptable carrier can also be selected on the basis of the desired route of administration. The desired route of administration may be one or more of buccal, oral, or enteral. For example, in a one embodiment the carrier is suitable for oral administration. In some embodiments, the composition includes a carrier or additional agent that is suitable for promoting delivery of the compound(s) to the gastrointestinal or intestinal tract. In additional embodiments, the composition is formulated for transdermal administration.

Pharmaceutical compositions intended for oral administration may be prepared according to any method known in the art for the production of pharmaceutical compositions, and such compositions may contain one or more agents selected from the group consisting of sweetening agents, flavoring agents, coloring agents, and preserving agents in order to provide pharmaceutically suitable and palatable preparations.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Solid dosage forms for oral administration include, but are not limited to, capsules, tablets, pills, powders, and granules. In such solid dosage forms, the active compounds are mixed with at least one pharmaceutically acceptable excipient or carrier such as, but not limited to, sodium citrate or dicalcium phosphate. Suitable carriers also include microcrystalline cellulose, rice bran, and silica, such as SILICA FLO GUARD SP™. Carriers can be used in combination.

The pharmaceutical composition can include fillers or extenders, such as starches, lactose, sucrose, glucose, mannitol, and silicic acid. Optionally, binders such as, for example, carboxymethylcellulose, alginates, gelatin, polyvinylpyrrolidone, sucrose, and acacia can be include. In additional embodiments, the pharmaceutical compositions include humectants, such as glycerol, and/or disintegrating agents, such as agar-agar, calcium carbonate, potato or tapioca starch, alginic acid, certain silicates, and sodium carbonate. The compositions can also include solution retarding agents such as paraffin, absorption accelerators such as quaternary ammonium compounds, wetting agents such as acetyl alcohol and glycerol mono stearate, absorbents such as kaolin and bentonite clay, and/or lubricants such as talc, calcium stearate, magnesium stearate, solid polyethylene glycols, sodium lauryl sulfate.

In the case of capsules, tablets and pills, the dosage form can also comprise buffering agents. Tablets may contain the active ingredient in admixture with non-toxic pharmaceutically acceptable excipients, which are suitable for the production of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations.

Pharmaceutical compositions suitable for oral administration can be presented in discrete units each containing a predetermined amount of at least one composition useful in the present methods; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the composition with a liquid or finely divided solid carrier, or both, and then, if necessary, shaping the product.

For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Compositions formulated for administration optionally contain opacifying agents and can also be of a composition that they release the active ingredient(s) in a certain part of the intestinal tract, optionally, in a delayed manner. Examples of embedding compositions which can be used include polymeric substances and waxes. The active compounds can also be in micro-encapsulated form, if appropriate, with one or more of the above mentioned excipients.

In some embodiments, solid dosage forms of tablets, dragees, capsules, pills, and granules can be prepared with coatings and shells such as enteric coatings and other coatings well known in the pharmaceutical formulating art. Oral delivery of the pharmaceutical compositions of the present disclosure can include compositions, as are well known in the art, to provide prolonged or sustained delivery of the drug to the gastrointestinal and/or intestinal tract by any number of mechanisms. These include, but are not limited to, pH sensitive release from the dosage form based on the changing pH of the small intestine, slow erosion of a tablet or capsule, retention in the stomach based on the physical properties of the composition, bioadhesion of the dosage form to the mucosal lining of the intestinal tract, or enzymatic release of the active drug from the dosage form. For some of the compositions, the intended effect is to extend the time period over which the active agents are is delivered to the site of action by manipulation of the dosage form. Thus, enteric-coated controlled release compositions are within the scope of the present disclosure. Suitable enteric coatings include cellulose acetate phthalate, polyvinylacetate phthalate, hydroxypropylmethylcellulose phthalate and anionic polymers of methacrylic acid, and methacrylic acid methyl ester. For example, a time delay material such as glyceryl monostearate or glyceryl distearate may be employed.

Compositions for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, any of a variety of herbal extracts, milk, olive oil, or other plant oil.

Liquid dosage forms for oral administration include, but are not limited to, pharmaceutically acceptable emulsions, solutions, suspensions, syrups, teas, and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A drinkable tea can also be used in the present methods. A drinkable tea may be taken in a liquid form or in a once pulverized or granulated form together with water or hot water. When it is in a powdery or granular form, the drinkable tea may be contained in a cavity of mouth before taking hot water or water like the conventional powdery or granular drinkable tea, or it may be taken after once dissolving in hot water or water. One or more components, such as a sugar, mint, or other flavor, can be added to improve taste and easiness as a drinkable drug. Teas, syrups and elixirs can be formulated with sweetening agents, for example glycerol, sorbitol or sucrose. Such compositions can also contain a demulcent, a preservative, and flavoring and coloring agents.

Aqueous suspensions can be produced that contain the active materials in admixture with excipients suitable for the production of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and 30 gum acacia; dispersing or wetting agents may be naturally occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable or plant oil, for example arachis oil, olive oil, sesame oil, broccoli oil, or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically, such co-solvents are employed at a level of from 0.01% to 2% by weight.

The disclosed compositions can also be used topically. In several embodiments, the method includes topical application, using an ointment, or transdermal application such as using a patch. For treatment of the skin, a therapeutically effective amount of at the composition can be locally administered to the affected area of the skin, such as in the form of an ointment. In one embodiment, the ointment is an entirely homogenous semi-solid external agent with a firmness appropriate for easy application to the skin. Such an ointment can include fats, fatty oils, lanoline, Vaseline, paraffin, wax, hard ointments, resins, plastics, glycols, higher alcohols, glycerol, water or emulsifier and a suspending agent. Using these ingredients as a base, a decoy compound can be evenly mixed. Depending on the base, the mixture can be in the form of an oleaginous ointment, an emulsified ointment, or a water-soluble ointment oleaginous ointments use bases such as plant and animal oils and fats, wax, Vaseline and liquid paraffin. Emulsified ointments are comprised of an oleaginous substance and water, emulsified with an emulsifier. They can take either an oil-in-water form (O/W) or a water-in-oil-form (W/O). The oil-in-water form (O/W) can be a hydrophilic ointment. The water-in-oil form (W/O) initially lacks an aqueous phase and can include hydrophilic Vaseline and purified lanoline, or it can contain a water-absorption ointment (including an aqueous phase) and hydrated lanoline. A water-soluble ointment can contain a completely water-soluble Macrogol base as its main ingredient.

Pharmaceutically acceptable carriers include a petroleum jelly, such as VASELINE®, wherein the petroleum jelly contains 5% stearyl alcohol, or petroleum jelly alone, or petroleum jelly containing liquid paraffin. Such carriers enable pharmaceutical compositions to be prescribed in forms appropriate for consumption, such as tablets, pills, sugar-coated agents, capsules, liquid preparations, gels, ointments, syrups, slurries, and suspensions. When locally administered into cells in an affected area or a tissue of interest, the composition can contain a synthetic or natural hydrophilic polymer as a carrier. Examples of such polymers include hydroxypropyl cellulose and polyethylene glycol. The disclosed composition can be mixed with a hydrophilic polymer in an appropriate solvent. The solvent is then removed by methods such as air-drying, and the remainder is then shaped into a desired form (for example, a sheet) and applied to the target site. Formulations containing such hydrophilic polymers keep well as they have a low water-content. At the time of use, they absorb water, becoming gels that also store well. In the case of sheets, the firmness can be adjusted by mixing a polyhydric alcohol with a hydrophilic polymer similar to those above, such as cellulose, starch and its derivatives, or synthetic polymeric compounds. Hydrophilic sheets thus formed can be used. A therapeutically effective amount of the disclosed composition can also be incorporated into bandages and dressings for wounds.

The above considerations concerning effective compositions and administration procedures are well known in the art and are described in standard textbooks. See e.g. Gennaro, A. R., Remington: The Science and Practice of Pharmacy, 15th Edition, (Lippincott, Williams and Wilkins), 2000; Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

### Methods of Use

The disclosed compositions are of use for increasing mitochondrial mass in a cell of a subject. These methods can include administering to the subject an effective amount of any of the disclosed compositions. The cells can be any cells of interest, such as, but not limited to, white blood cells such as lymphocytes, polymorphonuclear cells and/or monocytes.

In some embodiments, the disclosed compositions increase mitochondrial biogenesis. In some embodiments of the method, a subject is selected who is in need of increased mitochondrial biogenesis and/or antioxidant activity, and the composition is administered to the subject to increase any of these activities.

In some embodiments, the compositions disclosed herein are of use for activating the Nuclear Respiratory Factor (Nrf)-1 and Nrf-2 pathways, activating peroxisome proliferator-activated receptor γ coactivator 1 (PGC1) pathway, and/or increases phospholipase, cyclooxygenase and/or lipooxygenase pathways in the cell. The compositions disclosed herein also are of use for inhibiting the release or production of an interleukin, leukotriene, tumor necrosis factor, C-reactive protein, and/or an inflammatory cytokine. In some embodiments, the interleukin is interleukin (IL)-2, IL-4, IL-6, IL-8, or IL-10. In other embodiments, the tumor necrosis factor is tumor necrosis factor alpha. In further embodiments, the inflammatory cytokine is ganulocyte macrophage colony stimulating factor (GM-CSF) or interferon (IFN)-gamma.

In some embodiments, methods are disclosed herein for increasing the activity level, exercise capacity or stamina of a subject. The method can also include measuring the activity level and/or exercise capacity of the subject. In additional embodiments, methods are disclosed for increasing the alertness and cognition of a subject.

In other embodiments, the compositions promote healthy aging and/or avoids mitochondrial dysfunction. In some examples, the composition enhances cardiovascular health and/or brain health, improves metabolic function, decreases the likelihood of developing diabetes, and enhances stem cell renewal. Any of the disclosed compositions are of use in these methods. The disclosed compositions can also be used for promoting/increasing heart health, brain health, cognition/focus, and energy. The disclosed composition are of use for antioxidant protection, promoting healthy aging, as anti-aging compounds, and can be used to treat chronic disease.

The subject can be any subject of interest, including human and veterinary subjects. The subject can be human, including adults and children. The human subject can be a young adult (e.g., about 20 to about 35 years of age), middle-aged (e.g., about 40 to about 64 years of age), or elderly (over 65 years of age) subject.

The subject can be a healthy subject. Thus, in one embodiment, a subject is selected that does not have a pathological condition. The subject can be a subject for whom increased exercise capacity is desirable, such as an athlete or military personnel. The subject can be a subject for whom increased alertness and/or cognition is desirable, such as a pilot, military personnel, or an elderly subject. The subject can be a student, such as a college student.

The subject can have a disease, such as Alzheimer's disease, Parkinson's disease, Huntington's disease, or diabetes mellitus. The subject can have a hyperproliferative disorder, such as cancer (benign or malignant), psoriasis, amyotrophic lateral sclerosis (ALS), Friedreich's ataxia, or exercise intolerance. The cancer can be any cancer of interest, including solid or a hematologic tumor. In one specific non-limiting example, the cancer is colon cancer. The subject can have an autoimmune disorder, such as systemic lupus erythematosus (SLE), chronic fatigue syndrome, fibromyalgia or rheumatoid arthritis (RA). The subject can have a thyroid disorders, such as hypothyroidism. The subject can have heart disease, such as congestive heart failure, cardiomyopathy or a myocardial infarction. The subject can have a stroke. The subject can have diabetes.

In some embodiments, the disclosed compositions are of use to treat heart disease, such as congestive heart failure, cardiomyopathy, myocardial infarction, hypertension, and/or hypercholesterolemia. In other embodiments, the disclosed compositions are of use to treat stroke. In further embodiments, the disclosed compositions are of use to treat diabetes and related conditions (for example, diabetic neuropathy). The diabetes can be type I or type II diabetes. In yet other embodiments, the disclosed compositions are of use to treat a mitochondrial disorder. In additional embodiments, the disclosed compositions are of use to treat an autoimmune disease, such as SLE, chronic fatigue syndrome, RA or fibromyalgia). In further embodiments, the disclosed compositions are of use to treat hypothyroidism.

In some embodiments, the subject can have a mitochondrial disease, which are those disorders that affect the function of the mitochondria. Generally these diseases are due to disorders of oxidative phosphorylation. Mitochondrial diseases are often cause by a pathogenic mutation in a mitochondrial gene. The mutations are usually heteroplasmic so there is a mixture of normal and mutant DNA, the level of which can differ among tissues. In one embodiment, the subject has a mitochondrial disease, such as Leber's hereditary optic neuropathy (LHON), Leigh's disease (also known as Subacute Necrotizing Encephalomyelopathy), Neuropathy Ataxia and Retinitis Pigmentosa (NARP), or Myoneurogenic gastrointestinal encephalopathy (MNGIE).

The disclosed composition can be administered at selected time intervals over a treatment course of selected duration. The composition can be administered four times daily, thrice daily, twice daily, daily, bi-weekly, weekly or monthly. In one embodiment, the composition is administered daily. In another embodiment, the composition is administered twice daily. In an additional embodiment, the composition is administered with meals. In a specific, non-limiting example, the composition is administered twice daily with meals. In some embodiments, a double dose or a triple dose of the composition is administered. Thus, a double dose or triple dose can be administered four times daily, thrice daily, twice daily, daily, bi-weekly, weekly or monthly. In yet other embodiments, a double dose of the composition is administered once, twice or thrice daily with meals.

The subject can be treated over an extended period, such as for weeks, months or years. Specific examples include, but are not limited to, administration for a week, about ten days, about two weeks, about a month, about three months, about six months, about nine months, about a year, about two years, about three years, about five years, about ten years, or for the remaining lifespan of the subject.

The method can include performing an assay to determine mitochondrial mass in a cell. Mitochondrial mass can be evaluated using a mitochondria selective agent, such as a fluorescent agent. In some embodiments, mitochondrial mass is determined using nonylacridine orange. In other embodiments, mitochondrial mass is determined by morphometric analysis. In further, the number of mitochondria per cell in the sample is measured.

Methods for quantifying mitochondrial mass, volume and/or mitochondrial number are known in the art, and may include, for example, quantitative staining of a representative biological sample. Typically, quantitative staining of mitochondrial can be performed using organelle-selective probes or dyes, including but not limited to mitochondrion selective reagents such as fluorescent dyes that bind to mitochondrial molecular components (e.g., nonylacridine orange, MITOTRACKERS™, or potentiometric dyes that accumulate in mitochondria as a function of mitochondrial inner membrane electrochemical potential (see, e.g., Haugland, 1996 Handbook of Fluorescent Probes and Research Chemicals—Sixth Ed., Molecular Probes, Eugene, Oreg.). As another example, mitochondrial mass, volume and/or number may be quantified by morphometric analysis (see Cruz-Orive et al., 1990 Am. J. Physiol. 258:L148; Schwerzmann et al., 1986 J. Cell Biol. 102:97). These or any other means known in the art for quantifying mitochondrial mass, volume and/or mitochondrial number in a sample can be used.

The methods can also include performing a bioenergetics assay on a sample from the subject, such as a white blood cell or platelet sample from the subject. The bioenergetics assay can measure, for example, respiratory rate, reserve respiratory capacity, or ATP linked respiration of mitochondria in the platelet sample or white blood cell sample. The method can also include measuring mitochondrial function. In certain embodiments, an indicator of mitochondrial function is measured, such as a mitochondrial electron transport chain enzyme, a Krebs cycle enzyme, a mitochondrial matrix component, a mitochondrial membrane component or an ATP biosynthesis factor. In other embodiments, the indicator of mitochondrial function comprises free radical production. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the relative or absolute amount of mitochondrial DNA per cell in the patient.

The synthesis of ATP via oxidative phosphorylation is the most common function of mitochondria, and is a process is typically determined indirectly through measurement of mitochondrial oxygen (O₂) consumption, or respiration. Altered respiratory kinetics in response to specific substrates are often interpreted as reflecting changes in oxidative phosphorylation and the regulation of cellular energy homeostasis. According to Perry et al. (Diabetes 62: 1041-1053, 2013, incorporated herein by reference), the rate of mitochondrial respirometric O₂ flux can be a measured by several methods in the art. These methods include O₂-dependent quenching of porphyrin-based phosphors and amperometric O₂ sensors. Phosphorescent probes have also been used for in vitro/in situ respiratory measurements (e.g., the Seahorse Bioscience XF Extracellular Flux Analyzer or Luxcel MitoXpress). An amperometric approach is also of use.

In some examples, a Clark electrode can be utilized. A Clark electronde contains a gold or platinum cathode and an Ag/AgCl anode separated by a KCl solution. A voltage is applied to the two half-cells, which are separated from the experimental assay media by a membrane of O₂-permeant material (e.g., polyvinylidene difluoride). O₂ diffuses from the assay media through the membrane and is reduced by electrons at the cathode, yielding hydrogen peroxide (H₂O₂). The H₂O₂ then oxidizes the Ag of the Ag/AgCl anode, which generates an electrical current proportional to the partial pressure, and in turn concentration, of O₂ in the experimental solution. Changes in [O₂] in the assay media (typically 1-2 mL) therefore correspond to the inverse of the respiratory rate of a biological sample (e.g., mitochondria) and allow for quantification of O₂ consumption (Perry et al., supra).

The XF Extracellular Flux Analyzer (Seahorse Bioscience) can be used for measurement in intact adherent cells in culture. This system uses a 24- or 96-well cell culture microplate format that is mated from above with a sensor cartridge containing an equivalent number of individual probes, each containing fluorophores sensitive to O₂ or H⁺ embedded within the polymer that comprises the probe. A piston-like sensor cartridge is introduced periodically into the wells of the microplate, forming transient microchambers just above (˜200 microns) the cell monolayer. Fiber optic bundles inserted simultaneously by the machine into the probes of the sensor cartridge provide the excitation and collect the emission light for each fluorophore. The change in O₂ and H⁺ concentration in the media (7-10 μL) is measured over several minutes, reflecting the rate of cellular O₂ consumption and H⁺ production. Raising the sensor cartridge then allows the media above the cells to mix/reequilibrate. The XF Extracellular Flux Analyzer can also measure extracellular acidification rates and carbon dioxide evolution rates as indices of glycolysis and tricarboxylic acid cycle (TCA) kinetics (Perry et al., supra).

Mitochondria regulate cellular redox homeostasis through the establishment of the redox circuitry (e.g., via the NADPH/NADP couple) and emission of reactive oxidants. Reactive O₂ species (ROS) are molecules that are chemically reactive due to the incomplete reduction of O₂ within the molecule. These include free radicals possessing an unpaired electron such as the superoxide anion (O₂.⁻), hydroxyl radical (HO), peroxyl and nitroxyl radicals, as well as nonradical oxidants such as H₂O₂. Thus, the disclosed methods can include measurement of ROS.

Electron spin resonance/electron paramagnetic resonance can be used to measure free radicals, an approach that can be used in vivo under physiological conditions. A variation on the technique, known as spin trapping, is also frequently used; it incorporates a detecting molecule that reacts with, but is considerably more stable than, the initial free radical. The resulting free radical product often has spectral properties that allow the original radical to be identified indirectly. Although clinical applications continue to develop. Several fluorescent-, chemiluminescent-, and electrochemical/nanoparticle-based approaches are known in the art for the detection of oxidants. These include 2′,7′-dichlorodihydrofluorescein, triphenylphosphonium hydroethidine-based probe MITOSOX™ Red, and AMPLEX® Red (N-acetyl-3,7-dihydroxyphenoxazine, detects H2O2), see Perry et al, supra.

Mitochondrial oxidant production can also be measured. These methods are known in the art, and can utilize substrates in combination with inhibitors to specific sites within the respiratory complexes have been used to identify the sites and topology of O₂.⁻ production.

Membrane potential also can be measured. In some examples, cationic fluorescent probes can be used to qualitative changes in mitochondrial membrane potential in cultured cells using confocal microscopy and/or flow cytometry.

ATP production rate can also be measured. ATP production provides an index of oxidative phosphorylation activity in isolated mitochondria. The ATP produced is detected. In some examples, ATP production is detected through the reaction of luciferin with ATP, to ultimately generate oxyluciferin, AMP, and light via firefly luciferase, which is detected in a luminometer.

The sensitivity of the permeability transition pore to calcium loading has been used as an index of mitochondrial viability and can be detected. In some embodiments, this is evaluated by determining the calcium retention capacity of mitochondria to CALCIUM GREEN™-5N (Molecular Probes), a relatively low-affinity impermeable calcium indicator that exhibits increased fluorescence emission intensity upon binding calcium. Pulses of calcium are added at defined intervals, each eliciting a spike in signal that dissipates due to the uptake of calcium into the mitochondrial matrix. Calcium concentration can be monitored continuously with each addition until opening of the Permeability Transition Pore collapses the membrane potential and releases the accumulated calcium from the matrix, causing a sudden and sustained increase in signal. The amount of calcium retained prior to opening of the Permeability Transition Pore is considered an index of mitochondrial viability. Additional methods are disclosed in Perry et al. supra, see //diabetes.diabetesjournals_org/content/62/4/1041.full)

### EXAMPLES

Disclosed herein are compositions that increase the mitochondrial biomass in a cell, and are of use for increasing energy and cognition. The combination of ingredients produces a synergistic effect. These compositions include a unique combination of naturally occurring ingredients that provides these effects.

### Example 1

### Exemplary Test Product

The following test product, called “MEF-2,” was produced.

| Item Description | | Active | unit |
|---|---|---|---|
| 1 | Vitamin B2 Riboflavin | 12.5 | mg |
| 2 | Niacinamide | 50 | mg |
| 3 | Co Q 10 | 50 | mg |
| 4 | Magnesium (Magnesium Krebs) | 25 | mg |
| 5 | PQQ Na2 (Disodium Salt of Pyrroloquinoline | 10 | mg |
| | Quinine) | | |
| 6 | Iron (from Albion Ferrochel) | 15 | mg |
| 7 | Copper Citrate 10% | 2 | mg |
| 8 | Manganese (as Manganese Citrate) | 2 | mg |
| 9 | Molybdenum (as Molybdenum Citrate Complex) | 0.1 | mg |
| 10 | Zinc (Citrate 32%) | 15 | mg |
| 11 | Selenium (as Selenomethionine) | 0.2 | mg |
| 12 | Microcrystalline Cellulose, FCC VE-090** | 100 | mg |
| 13 | Rice Bran** | 250 | mg |
| 14 | Silica Flo Guard SP** | 35 | mg |
| | | 566.8 | mg |

Italicized ingredients are carriers.

### Example 2

### Total Antioxidant Capacity

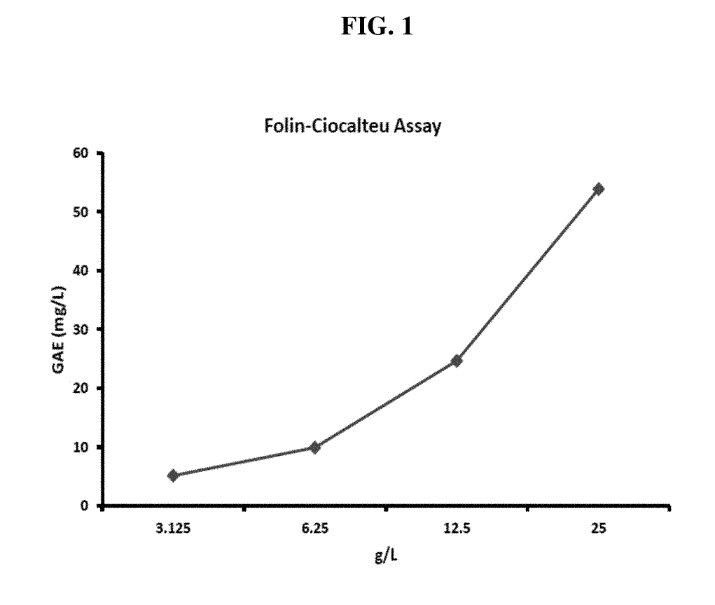

The MEF-2 test product was tested in the Folin-Ciocalteu assay (also known as the total phenolics assay). This assay makes use of the Folin-Ciocalteu reagent to measure antioxidants. The assay was performed by adding the Folin-Ciocalteu's phenol reagent to serial dilutions of extract, thoroughly mixing, and incubating for 5 minutes. Sodium carbonate was added, starting a chemical reaction producing a color. The reaction was allowed to continue for 30 minutes at 37° C. Optical absorbance was measured at 765 nm in a colorimetric plate reader. Gallic acid was used as a reference standard, and the data reported in Gallic Acid Equivalents per gram product. The antioxidant capacity was good, and correlated with the amount used in the experiments, see FIG. 1.

### Example 2

### Mitochondrial Mass

White blood cells (leukocytes) were obtained from a healthy human blood donor. Serial dilutions of the MEF-2 test product were added to cell cultures and mitochondrial mass was measured after 2 hours incubation. Untreated control samples were processed in parallel. After incubation, cells were stained with the fluorescent probe MITOTRACKER® Green that stains mitochondria in proportion to mitochondrial mass per cell. The overall sum of fluorescent output per cell is a measure of mitochondrial mass per cell. The ATTUNE® acoustic aligning flow cytometer allows the separate analysis of white blood cell subsets and provides fluorescence intensity measurements per cell, where the fluorescence intensity is proportional to the mitochondrial mass per cell.

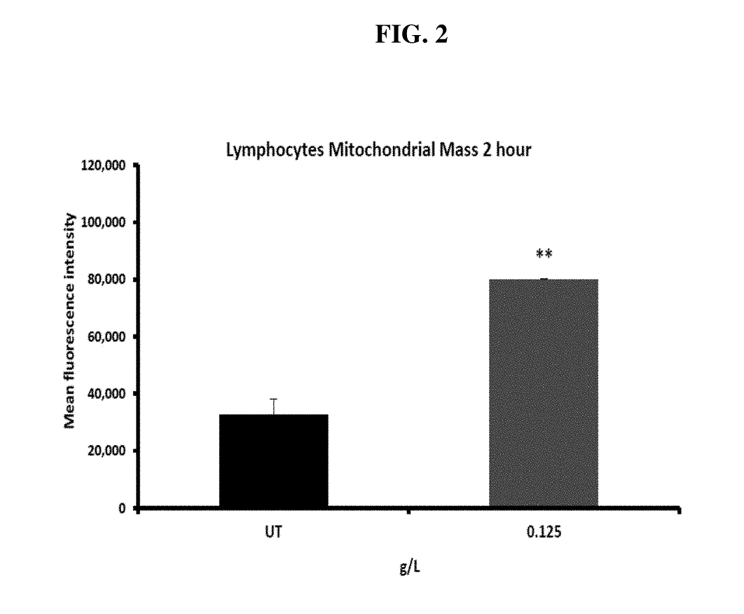

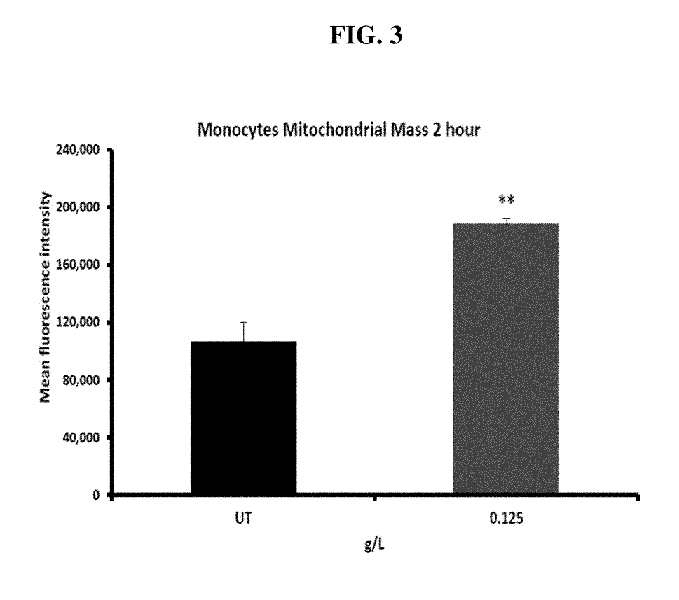

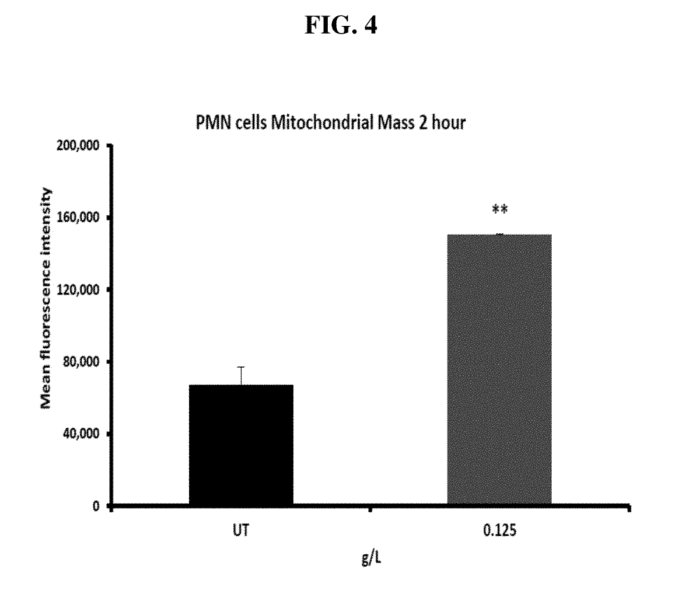

The test product, MEF-2, increased mitochondrial mass in two hour cultures. Treatment of cultures with the 0.125 g/L dose of MEF-2 led to a large increase in mitochondrial mass (80-140%) in all three white blood cell populations (see FIG. 2-4). Overall, a robust change in mitochondrial mass per cell was seen in three different white blood cell populations.

### Example 3

### Clinical Trial

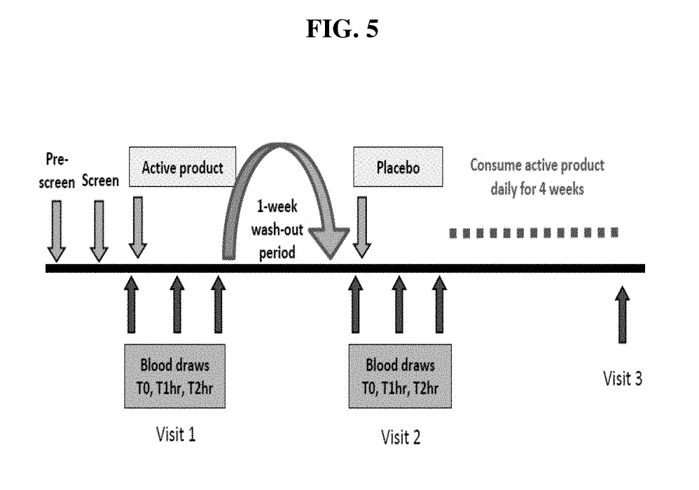

A study protocol is shown in FIG. 5. Upon arrival on the morning of each clinic day, participants will rest in a seated position quietly for 1 hour prior to baseline blood draw. This resting period is crucial to gain representative baseline data. During this time, questionnaires will be completed to monitor previous meals, snacks, exercise, stressors, and recent sickness. The baseline blood draw will be followed immediately by feeding a product to the volunteer, who will continue to rest quietly. One more blood draw is taken at 2 hours after consumption. Blood pressure and responses to a brief questionnaire on mental state are recorded prior to each blood draw.

### Inclusion Criteria

Healthy adults 50-70 years of age;
BMI between 25-40;
Veins easy to see in both arms.

### Exclusion Criteria

Previous major gastrointestinal surgery (absorption of test product may be altered)
(minor surgery not a problem);
Taking daily OTC medications (NSAIDS, allergy medications, and others) (birth control not a problem);
Taking anti-depressants or hypnotics;
Currently experiencing intense stressful events and life changes;
Actively depressed;
Experiencing sleep disturbances;
Working night shift;
Pregnant, nursing, or trying to become pregnant;
Food intolerances or allergies currently causing discomfort (such as Celiac disease), due to ongoing inflammatory reactions that may negatively affect product absorption within the 2 hours of testing;
Food allergies related to ingredients in test product.

### Mitochondrial Testing of Blood Cells

The testing described below will be performed on polymorphonuclear and mononuclear cells from blood samples taken before consumption and at 2 hours, and 4 weeks after consumption.

### Mitochondrial Mass Per Cell

Cells will be stained with a fluorescent probe that stains mitochondria in proportion to mitochondrial mass per cell. The ATTUNE® acoustic aligning flow cytometer tracks cell numbers per volume unit, and provides fluorescence intensity measurements per cell. The overall sum of fluorescent output per cell reflects mitochondrial mass per cell.

Mitochondrial Function

In addition, polymorphonuclear and mononuclear cells will be tested for mitochondrial function using the MTT assay. The use of the MTT assay for this project will be exploratory, as it has not previously been used in clinical studies. Freshly harvested cells from the blood samples from study participants will be cultured for 4 hours to allow the color formation to take place in proportion to mitochondrial function. In the MTT bioassay, chemical reactions trigger a specific color development based on cellular functions:
When a reduction in color is measured, this is linked to a reduced cellular viability, either as a result of direct killing, or inhibition of mitochondrial function leading to cell death.
When an increase in color is measured, this has two possible explanations: 1) Increased cell numbers (growth); 2) increased mitochondrial mass, and 3) increased mitochondrial function (energy production).

### Safety Testing

At the initial baseline visit and at the study exit visit, extra blood will be drawn to allow for testing for basic safety parameters. This will include a Complete Blood Count with Differential Count (CBC) and a comprehensive metabolic panel for blood chemistry (CMP). The purpose of this testing is to help build the safety portfolio for the product.

### Table Showing Study Procedures:

| | | | Baseline | 2 hour | Baseline | 2 hour | 4 weeks |
|---|---|---|---|---|---|---|---|
| | Pre- | | Study day 1 | | Study day 2 | | Study day 3 |
| | screen | Screen | PLACEBO-CONTROLLED | | PLACEBO-CONTROLLED | | OPEN-LABEL |
| PHYSICAL | | | | | | | |
| Medical history | X | | | | | | |
| Health status interview | X | | | | | | X |
| Informed consent | | X | | | | | |
| Height | | X | | | | | |
| Weight (BMI) | | X | X | | | X | X |
| Blood pressure | | | X | X | X | X | X |
| BLOOD TESTS | | | | | | | |
| Pregnancy test (females) | | X | | | | | |
| CBC | | | X | | | | X |
| CMP | | | X | | | | X |
| C-reactive protein, high sensitivity | | | X | X | X | X | X |
| Mitochondrial mass per cell* | | | X | X | X | X | X |
| Mitochondrial function (MTT) | | | X | X | X | X | X |
| Inflammatory cytokine panel 8-plex | | | X | X | X | X | X |
| Serum banking** | | | X | X | X | X | X |
| QUESTIONNAIRES | | | | | | | |
| Fatigue Assessment Inventory | | | X | | X | | X |
| Wellness | | | X | | X | | X |
| Current medications | | | X | | X | | X |
| Current supplements | | | X | | X | | X |
| Compliance | | | | | | | X |
| Adverse events | | | | | X | | X |

*Mitochondrial volume per cell will be determined on freshly drawn blood samples from subjects. Staining will be performed on freshly stained white blood cells using a fluorescent dye for mitochondria, and analyzed by multi-parameter flow cytometry on-site. This will avoid sample storage and shipment, and will assure that all testing of mitochondrial function is performed on-site same-day on each blood sample.
**The banking of serum samples allows decisions to be made at a later time on whether additional biomarkers are beneficial, without having to repeat the clinical study.

### Additional Assays

Cellular Antioxidant Protection Using the CAP-e Bioassay;
Antioxidant capacity using the Ferric-reducing Antioxidant Potential (FRAP) test;
Extensive cytokine profile (pro- and anti-inflammatory biomarkers). The cytokine profile is: cytokine panel is: IL-1beta, IL-1ra, IL-2, IL-4, IL-5, IL-6, IL-7, IL-8, IL-9, IL-10, IL-12 (p′70), IL-13, IL-15, IL-17, eotaxin, basic FGF, G-CSF, GM-CSF, IFNgamma, IP-10, MCP-1 (MCAF), MIP-1alpha, MIP-1beta, PDGF-BB, RANTES, TNF-alpha, and VEGF.

A single dose of Mitochondrial Energy Formula, at the recommended daily dose, produces quantifiable changes in mitochondrial volume.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A composition comprising formulation A, wherein formulation A comprises
pyrroloquinoline quinone (PQQ), coenzyme Q (CoQ10), niacinamide and riboflavin in a combined amount sufficient to increase mitochondrial mass in a cell.

2. The composition of claim 1, comprising about 5% to about 8% PQQ by weight percent of formulation A.

3. The composition of claim 1, comprising about 41% to about 43% CoQ10 by weight of formulation A.

4. The composition of claim 1, comprising about 41% to about 43% of niacinamide by weight of formulation A.

5. The composition of claim 1, comprising about 10% riboflavin by weight of formulation A.

6. The composition of claim 1, comprising PQQ, CoQ10, niacinamide, and riboflavin at a ratio of about 1:5:5:1.2, respectively.

7. The composition of claim 1, comprising:
about 5 to about 20 milligrams (mg) of PQQ;
about 45 to about 100 mg of CoQ10;
about 45 to about 100 mg of niacinamide; and
about 10 to about 25 mg of riboflavin.

8. The composition of claim 7, comprising
about 10 mg of PQQ;
about 50 mg of CoQ10;
about 50 mg of niacinamide;
about 12.5 mg of riboflavin.

9. The composition of claim 7, further comprising one or more of a magnesium chelate, iron, a copper salt, a manganese salt, a molybdenum salt, a zinc salt, and organic selenium, in an amount sufficient to increase mitochondrial function.

10. The composition of claim 9, comprising:
about 20 to about 50 mg of the magnesium chelate;
about 10 to about 30 mg of iron;
about 1 mg to about 4 mg of copper salt;
about 1 mg to about 4 mg of manganese salt;
about 0.05 to about 2 mg of the molybdenum salt;
about 10 mg to about 30 mg of a zinc salt; and/or
about 0.1 mg to about 0.4 mg of organic selenium.

11. The composition of acclaim 10, comprising
about 25 mg of the magnesium chelate;
about 15 mg of iron;
about 2 mg of copper salt;
about 2 mg of manganese salt;
about 15 mg of a zinc salt;
about 0.1 mg of the molybdenum salt; and/or
about 0.2 mg of organic selenium.

12. The composition of claim 9, further comprising formulation B, wherein formulation B comprises the magnesium chelate, the iron, the copper salt, the manganese salt, the molybdenum salt, the zinc salt, and the organic selenium.

13. The composition of claim 10, wherein the organic selenium is selenomethionine or selenocysteine.

14. The composition of claim 12, wherein formulation B comprises:
about 42% to about 47% of the magnesium chelate by weight of formulation B;
about 23% to about 25% of the iron by weight of formulation B;
about 2% to about 3% of the copper salt by weight of formulation B;
about 2% to about 3% of the manganese salt by weight of formulation B;
about 0.1% to about 0.2% of the molybdenum salt by weight of formulation B;
about 23% to about 25% of the zinc salt by weight of formulation B; and
about 0.2% to about 0.3% of the organic selenium by weight of formulation B.

15. The composition of claim 12, comprising:
about 20 to about 50 mg of the magnesium chelate;
about 10 to about 30 mg of iron;
about 1 mg to about 4 mg of copper salt;
about 1 mg to about 4 mg of manganese salt;
about 0.05 to about 2 mg of the molybdenum salt;
about 10 mg to about 30 mg of a zinc salt; and
about 0.1 mg to about 0.4 mg of organic selenium.

16. The composition of claim 12, comprising
about 25 mg of the magnesium chelate;
about 15 mg of iron;
about 2 mg of copper salt;
about 2 mg of manganese salt;
about 15 mg of a zinc salt;
about 0.1 mg of the molybdenum salt; and
about 0.2 mg of organic selenium.

17. The composition of claim 9, wherein the copper salt is copper citrate.

18. The composition of claim 9, wherein the manganese salt is manganese citrate.

19. The composition of claim 9, wherein the molybdenum salt is molybdenum citrate.

20. The composition of claim 9, wherein the zinc salt is zinc citrate.

21. The composition of claim 1, further comprising a pharmaceutically acceptable carrier.

22. A composition, comprising all of:
about 5 to about 20 milligrams (mg) of PQQ;
about 45 to about 100 mg of CoQ10;
about 45 to about 100 mg of niacinamide;
about 10 to about 25 mg of riboflavin;
about 20 to about 50 mg of the magnesium chelate;
about 10 to about 30 mg of iron;
about 1 mg to about 4 mg of copper salt;
about 1 mg to about 4 mg of manganese salt;
about 0.05 to about 2 mg of the molybdenum salt;
about 10 mg to about 30 mg of a zinc salt;
about 0.1 mg to about 0.4 mg of organic selenium;
and a pharmaceutically acceptable carrier.

23. The composition of claim 22, comprising:
about 10 mg of PQQ;
about 50 mg of CoQ10;
about 50 mg of niacinamide;
about 12.5 mg riboflavin
about 25 magnesium chelate
about 15 mg iron
about 2 mg copper citrate
about 2 mg of manganese citrate
about 0.1 mg of molybdenum citrate;
about 15 mg of zinc citrate; and
about 0.2 of selenomethionine.

24. A method of increasing mitochondrial mass in a cell of a subject, comprising administering to the subject an effective amount of the composition of claim 1, thereby increasing mitochondrial mass in the cell of the subject.

25. A method of increasing mitochondrial biogenesis in a cell of a subject, comprising administering to the subject an effective amount of the composition of claim 1, thereby increasing mitochondrial biogenesis in the cell of the subject.

26. A method of increasing the activity level, exercise capacity or stamina of a subject, comprising administering to the subject an effective amount of the composition of claim 1, thereby increasing the activity level, exercise capacity or stamina of the subject.

27. A method of increasing the alertness and cognition of a subject, comprising administering to the subject an effective amount of the composition of claim 1, thereby increasing the alertness or cognition of the subject.

28. A method of improving mitochondrial function in a subject, comprising administering to the subject an effective amount of the composition of claim 1, thereby improving mitochondrial function.
